(19) United States
(12) Patent Application Publication
(10) Pub. No.: US 2018/0326111 A1
(43) Pub. Date: Nov. 15, 2018

(54) AEROSOL-FREE, SPILL-RESISTANT VOLATILE FLUID DISPENSING DEVICE

(71) Applicant: Microlin, LLC

(72) Inventor: Ashok V. Joshi, Salt Lake City, UT (US)

(21) Appl. No.: 16/046,963

(22) Filed: Jul. 26, 2018

Related U.S. Application Data
(63) Continuation-in-part of application No. 15/413,233, filed on Jan. 23, 2017.
Application No. 15/413,233 is a continuation-in-part of application No. 15/721,942, filed on Oct. 1, 2017.
Application No. 15/721,942 is a continuation-in-part of application No. 15/485,206, filed on Apr. 11, 2017.
Application No. 15/485,206 is a continuation-in-part of application No. 14/010,242, filed on Aug. 26, 2013, now Pat. No. 9,840,361.
Application No. 15/721,942 is a continuation-in-part of application No. 15/396,759, filed on Jan. 2, 2017.
Application No. 15/396,759 is a continuation-in-part of application No. 14/632,970, filed on Feb. 26, 2015, now Pat. No. 9,533,066.
Application No. 14/632,970 is a continuation-in-part of application No. 14/537,691, filed on Nov. 10, 2014, now Pat. No. 9,623,135.
(60) Provisional application No. 62/537,868, filed on Jul. 27, 2017.
(60) Provisional application No. 61/692,750, filed on Aug. 24, 2012.
(60) Provisional application No. 61/944,698, filed on Feb. 26, 2014.
(60) Provisional application No. 61/902,031, filed on Nov. 8, 2013.

(51) Int. Cl. A61L 9/12; B01F 3/04

(52) U.S. Cl. CPC A61L 9/12; B05B 11/02; B01F 3/04

(57) ABSTRACT

A device for introducing volatile fluid into a local environment. A volatile fluid chamber holds a quantity of volatile fluid in a solidified form to permit sustained release of volatile fluid in vapor form. A device may cause release of volatile fluid vapor into the local environment as a steady stream or intermittent puff. Gas discharge from the device may be urged by way of a vacuum pump, compressor, container of compressed gas, or by way of a gas-generating chemical reaction.

### RELATED APPLICATIONS

This application claims the benefit under 35 U.S.C. 119(e) of the filing date of Provisional Application Ser. No. 62/537,868, filed Jul. 27, 2017, for “AEROSOL-FREE, SPILL-RESISTANT VOLATILE FLUID DISPENSING DEVICE”; this application is also a continuation-in-part of U.S. patent application Ser. No. 15/413,233, titled “HIGH SURFACE AREA RESERVOIR FOR VOLATILE FLUID DISPENSER”, filed Jan. 23, 2017, which is a continuation-in-part of the International Application identified under Serial No. PCT/US2016/041007, titled “AIR FRESHENER WITH OPTIONAL DRAIN CLEANER” with an International filing date of Jul. 5, 2016, which is a continuation-in-part of U.S. patent application Ser. No. 14/792,332, filed Jul. 6, 2015, for AIR FRESHENER WITH OPTIONAL DRAIN CLEANER; this application is also a continuation-in-part of U.S. Utility patent application Ser. No. 15/721,942, filed Oct. 1, 2017 and titled “GAS CELL DRIVEN FLUID DELIVERY DEVICE FOR SPILL-RESISTANT STORAGE AND USE”, which is a continuation-in-part of U.S. Utility patent application Ser. No. 15/485,206, filed Apr. 11, 2017 and titled “SPILL-RESISTANT FLUID DELIVERY DEVICE”, which is a continuation-in-part of U.S. Utility patent application Ser. No. 14/010,242, filed Aug. 26, 2013 and titled “GAS CELL DRIVEN ORIENTATION INDEPENDENT DELIVERY DEVICE”, which claims the benefit of the filing date of U.S. Provisional Application Ser. No. 61/692,750, filed Aug. 24, 2012; application Ser. No. 15/721,942 is also is a continuation-in-part of U.S. Utility patent application Ser. No. 15/396,759, filed Jan. 2, 2017 and titled “NO-DRIP VOLATILE SUBSTANCE DELIVERY SYSTEM”, which is a continuation-in-part of U.S. Utility patent application Ser. No. 14/632,970, filed on Feb. 26, 2015, now U.S. Pat. No. 9,533,066, issued Jan. 3, 2017 and titled “VOLATILE SUBSTANCE DELIVERY SYSTEM, which claims the benefit of the filing date of U.S. Provisional Application Ser. No. 61/944,698, filed on Feb. 26, 2014, and is a continuation-in-part of U.S. Utility patent application Ser. No. 14/537,691, filed Nov. 10, 2014 and titled “VOLATILE SUBSTANCE DELIVERY SYSTEM”, which claims the benefit of the filing date of U.S. Provisional Application Ser. No. 61/902,031, filed on Nov. 8, 2013, the entire contents of all of which are incorporated by this reference as though set forth herein in their entirety.

### BACKGROUND

### Field of the Invention

This invention relates to dispensing devices for delivering one or more dose of volatile fluid to a local environment.

### State of the Art

It is sometimes desirable to dispense discrete doses of a volatile fluid for a beneficial purpose, such as to freshen-up air in a local environment from time-to-time. Dose discharge may be under manual control, or automated. It is known to provide containers for holding a bulk quantity of volatile fluid (e.g. a fragrance), and a dispensing mechanism structured to release individual doses of the volatile fluid from the container to the environment. A ubiquitous dispensing mechanism includes aerosol pressurized inside the container and a cooperating nozzle to discharge a mist or vapor of volatile fluid carried by the aerosol. Unfortunately, many convenient aerosols are now recognized as being damaging to the global environment, and their continued use is undesirable.

Containers for holding a bulk quantity of volatile fluid are subject to spilling, or accidental discharge, of the fluid. Accidental discharge of confined volatile fluid reduces the life of a volatile fluid-dispensing device. Many volatile fluids are damaging to materials that may encounter the accidentally discharged volatile fluid. It is desirable to provide a storage mechanism for volatile fluids that reduces risk of accidentally spilling (or otherwise emitting) quantities of the bulk fluid.

Various ways to manufacture devices in which to confine a quantity of volatile fluid are known. Volatile fluids can be very expensive. Certain manufacturing methods e.g., for fragrant items, are inherently inefficient at transfer of a volatile fluid (fragrance) into a fragrance-carrying device. One example is an injection molding operation, in which one or more volatile fragrance is heated along with plastic for injection of molten plastic (and entrained volatile fluid) into a mold to form a net-shape fragrant item. Large portions of the heated fragrance are typically off-gassed into the local manufacturing environment. These sorts of processes can be described as wasteful of volatile fragrance. It would be an improvement to provide volatile fluid-carrying devices, and one or more method to manufacture and/or to use those devices, which reduce waste of a volatile fluid during manufacture and/or use of the volatile fluid-carrying devices.

### BRIEF SUMMARY OF THE INVENTION

One aspect of this invention provides an apparatus for dispensing volatile fluid into a local atmosphere. A dose of volatile fluid may be dispensed by way of an intermittent puff or a continuous stream of gas.

The invention may be embodied to include a carrier material selected from the group consisting of styrene-based polymers, styrene-based rubbers, EPDM, butadiene-based polymers, gum rubber, cellulosic rubber, thermoplastic polyether- or polyester-based polymers and rubbers (TPE), thermoplastic urethane (TPU), an adsorbent material having high-surface area greater than about 10 m²/g, an absorbent cellulose or polymer sponge, a gas-generating chemical compound, and a mixture of two or more of the foregoing elements; and a volatile fluid dispersed inside the carrier material to a weight percent of between about 3% and about 400%, where weight percent is calculated as A/B*100, and A is weight of volatile fluid and B is weight of carrier material, to result in a spill-resistant solidified form of volatile fluid and to permit sustained release of volatile fluid vapor, wherein the volatile fluid is loaded into the carrier material at ambient temperature condition.

An exemplary device includes a volatile fluid chamber to hold a quantity of solidified volatile fluid. A solidified volatile fluid may be disposed inside the volatile fluid chamber, which may be refillable or replaceable. A workable volatile fluid chamber may be structured as a replaceable cartridge to couple with structure associated with the gas-moving system.

A workable volatile fluid chamber may be sized to hold solidified volatile fluid in an amount between about 10 ml and about 1.5 liters. Preferably, the volatile fluid is configured and arranged in a solidified form to permit sustained release of volatile fluid vapor to mix the vapor with a gas in the volatile fluid chamber.

A device typically also includes a gas discharge port to communicate a mix of volatile fluid vapor and gas from the volatile fluid chamber to permit discharge of volatile fluid in vapor form into the local atmosphere. Also, a gas-moving system is typically included to urge motion of gas from the volatile fluid chamber through the gas discharge port.

In certain cases, the gas-moving system may include a compressor disposed upstream of the volatile fluid chamber. In one alternative, a gas-moving system may include a vacuum pump disposed downstream of the volatile fluid chamber. It is within contemplation that a gas-moving system may include a transducer configured to operate on the solidified form to urge release of volatile vapor. A workable transducer may be selected from the group consisting of a heater, a nebulizer, and a microwave generator. Optionally, a gas-moving system may include a one-way valve disposed in a path of gas to resist flow of gas in a direction away from the discharge port. A workable gas-moving system may include a pressure relief valve or other mechanism or control arrangement to create distinct intermittent puffs of released volatile fluid.

A workable gas-moving system may even include a gas-generating material to increase a pressure inside the volatile fluid chamber as a result of a chemical reaction, and generated gas is used to propel volatile vapor into a local atmosphere. An exemplary gas-generating material includes a substrate selected from the group consisting of citric acid and soda, Magnesium hydride, Calcium hydride, Magnesium dioxide, Magnesium powder, Aluminum powder, Lithium metal, and Calcium metal. Any element or compound that produces gas upon contact with water or other injectable fluid is operable. In any case, the volatile fluid is dispersed into the carrier material substrate to form solidified volatile fluid.

A currently preferred gas-generating system further includes a water injection system configured and arranged to deliver water onto the substrate to cause gas generaion. A dose of applied water may be sized to generate a desired chemical reaction to produce a desired amount of gas. A water injection system may include a gas-generating cell arranged to increase pressure in a vessel of water for delivery of the water to the substrate. An alternative water injection system may include a mechanical water injector.

The invention may be embodied as a method. One method according to certain principles of the invention includes disposing a quantity of volatile fluid in a substrate to form solidified volatile fluid; and coupling the solidified volatile fluid to a gas-moving system for discharge of volatile vapor through a discharge orifice to a local environment. Preferably, the step of disposing volatile fluid in a substrate is performed at an ambient temperature. Sometimes, the gas-moving system may be operated at intervals to discharge discrete puffs of volatile fluid vapor to the local environment. Operation may be manual, or automated in some way.

One method according to certain principles of the invention includes providing a volatile fluid distribution system and operating the volatile fluid distribution system to discharge volatile fluid vapor through the discharge port and into the local atmosphere. A preferred volatile fluid distribution system includes a volatile fluid chamber to hold a quantity of volatile fluid. Desirably, the volatile fluid is configured and arranged in a solidified form to permit sustained release of volatile fluid vapor to mix the vapor with a gas in the volatile fluid chamber. A gas discharge port is disposed to communicate a mix of volatile fluid vapor and gas from the volatile fluid chamber to permit discharge of volatile fluid in vapor form into the local atmosphere. Also, a gas-moving system is provided to urge motion of gas from the volatile fluid chamber through the gas discharge port. It is desirable for the volatile fluid to be dispersed into a substrate at ambient temperature conditions to form the solidified form of volatile fluid.

### BRIEF DESCRIPTION OF THE DRAWINGS

In the drawings, which illustrate what are currently considered to be the best modes for carrying out the invention:

FIG. 1 is a schematic view of an operable device according to certain principles of the invention;

FIG. 2 is a schematic view of an operable device according to certain principles of the invention;

FIG. 3 is a schematic view of an operable device according to certain principles of the invention

FIG. 4 is a schematic view of an operable device according to certain principles of the invention; and

FIG. 5 is a schematic in side view illustrating a manufacturing process according to certain principles of the invention.

### DETAILED DESCRIPTION OF THE ILLUSTRATED EMBODIMENT(S)

Reference will now be made to the drawings in which the various elements of the illustrated embodiments will be given numerical designations and in which the invention will be discussed so as to enable one skilled in the art to make and use the invention. It is to be understood that the following description is only exemplary of certain principles of the present invention, and should not be viewed as narrowing the claims which follow.

Throughout this disclosure, it is within contemplation that one or more elements may be redacted in certain workable embodiments. Additional elements may be included in other embodiments. Any one or more of the various elements described and/or illustrated in connection with one device may be extracted and combined with other element(s) in a different arrangement to form alternative embodiments within the ambit of the invention. Although it is not currently preferred, even aerosol may be included in certain embodiments structured according to certain principles of the invention.

FIG. 1 illustrates a first currently preferred arrangement, generally indicated at 100, for dispensing volatile fluid into a local environment. A bulk container 102 is provided to hold a quantity of volatile fluid. Preferably, volatile fluid held in the bulk container 102 is stored in a solidized form, as described in detail below. In that case, risk of accidentally spilling the volatile fluid is virtually eliminated. A compressor or pump 104 may be coupled in fluid communication with the bulk container 102 to cause internal pressure in excess of local atmospheric pressure. The compressor 104 introduces gas (e.g., air 106), which essentially takes the place of an undesirable aerosol as a carrier for volatile fluid to the environment. Fluid communication through a device 100 may be provided by one or more stretch of conduit 108

Appropriate electronics and controls 110 are generally provided, in accordance with known devices, to permit operation of the compressor 104 as desired. Controls and electronics 110 can include an energy source, such as a battery or wired plug for an electric utility, a simple on/off switch, timer, and even local intelligence, such as a CPU or other digital controller.

As illustrated, one or more one-way valve 112 may be provided to control direction of gas flow through the device 100 for exit of volatile fluid-loaded gas 114 through a discharge orifice, generally 116. The pressurized gas (e.g., air) in the bulk container 102 inherently becomes loaded with volatile fluid over a time increment, and in certain embodiments, a pressure relief valve 118 may periodically emit a quantity of volatile fluid in vapor form 114. A preferred emission event may be characterized as a “puff”, generally 120. Operation of a compressor/pump 104 and pressure relief valve 118 are sometimes adjustable to fine-tune the device 100 for particular local conditions.

In an exemplary embodiment 100, the pressure relief valve 118 is set to maintain about 10 psig in the container. The compressor 104 is operated at intervals, or to slowly increase pressure in the container. The pressure relief valve 118 then intermittently releases a “puff” 120 of volatile fluid carried by air 114. Time increments between each “puff” 120 can be regulated as desired to accommodate a particular situation.

Alternative arrangements are within contemplation. For non-limiting examples, the illustrated pressure-relief valve 118 may be redacted, and the pump 106 may simply be energized at time of desired use to dispense volatile fluid in vapor form 114. It is currently preferred for volatile fluid emission to be performed at periodic times, as a “puff” 120. However, in some cases, the compressor 104 may be set to run for a sustained period, in which case the discharge 114 would more reasonably be characterized as a stream of gas including volatile fluid vapor. The one-way valve 112 may not be present in certain embodiments, or may be provided as an integral portion of a compressor 106.

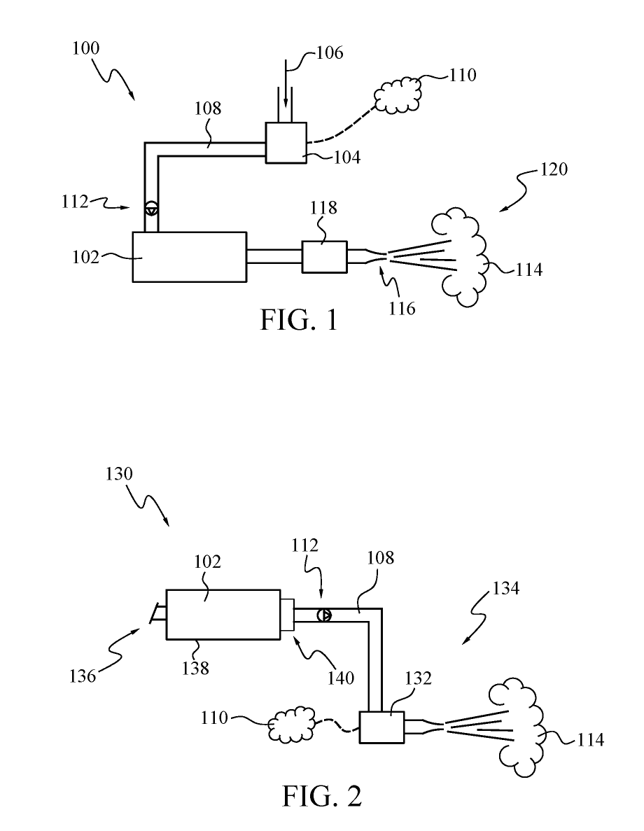

The embodiment indicated generally at 130 in FIG. 2 illustrates a bulk container 102 for holding volatile fluid, where a vacuum pump 132 is employed (in place of aerosol) to discharge volatile fluid vapor 114 into the environment. The vacuum pump 132 may be energized for a sustained period, or intermittently by a control system 110, similarly to the compressor 104 of FIG. 1.

A “puff” generator, generally 134, may be included, for intermittent discharge of volatile fluid vapor to the local environment. A puff generator 134 may be embodied in various ways, including electrical and/or mechanical arrangements effective to cause a timed release of gas from a device 130. As mentioned above, a pressure relief valve 118 is an operable puff generator. A pressure relief valve 118 may be an entirely mechanical structural arrangement to open at an elevated threshold pressure, and reseal at a lower set point pressure. Other sorts of valves may be employed in alternative arrangements. For non-limiting example, a solenoid can be placed in-circuit to release gas for a period of time that may be controlled, e.g., by a mechanical or electrical timer of some sort.

Still with reference to FIG. 2, the bulk container 102 may also include a vent, generally 136, to admit make-up air to replace gas that is evacuated by the vacuum 132. A workable vent 136 may be a mechanical valve, or a membrane that is porous to gasses, or other known device effective to admit gas. Sometimes, a one-way valve 112 may be included to control direction of gas flow through the device 130.

As also illustrated in FIG. 2, certain embodiments can be structured to permit replacement of spent bulk containers 102. The container 102 in FIG. 2 is embodied as a cartridge 138 that removably couples at connection structure, generally indicated at 140, for communication of volatile fluid/vapor to the vacuum pump 132.

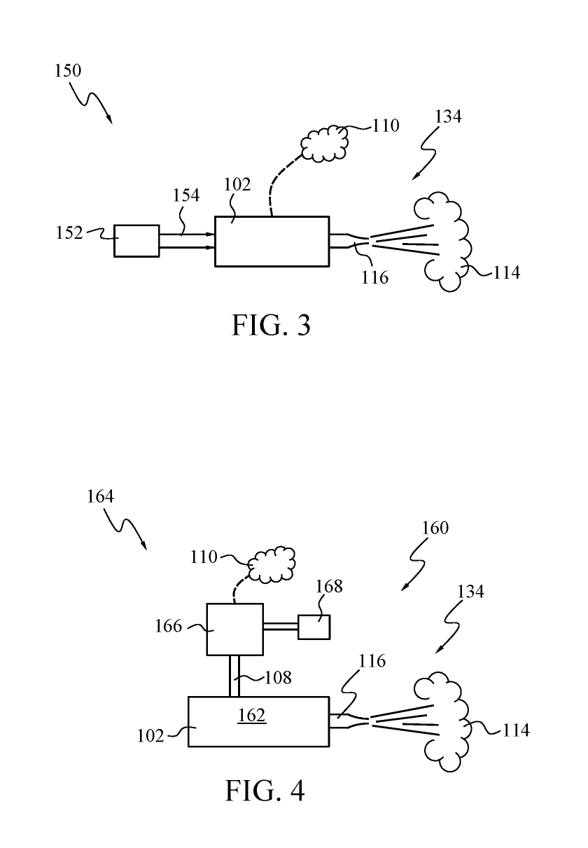

The device indicated generally at 150 in FIG. 3 illustrates a bulk container 102 for volatile fluid where the container 102 is coupled to a transducer 152 to facilitate evolution of volatile fluid gas from a carrier material disposed inside the container 102. Exemplary transducers 152 include a microwave generator, heater, fan, and/or a nebulizer. Various connection structure 154 may be envisioned, including mechanical linkages, line-of-sight, and ducting. One or more transducer(s) may be external and/or internal to the container 102. For example, when the volatile fluid is in solidized form, a nebulizer may be configured and arranged in one of various workable arrangements to shake the carrier and/or its confined volatile fluid storage media at a frequency effective to promote release of volatile fluid vapor. A control mechanism 110 may be provided to cause discharge of volatile vapor or mist at a desired rate and/or frequency.

The embodiment indicated generally at 160 in FIG. 4 is configured to generate gas to propel volatile fluid into a local environment. The bulk container 102 holds a quantity of solidized volatile fluid 162 structured to generate gas on demand. A workable solidized volatile fluid includes a volatile fluid distributed into a substrate that includes a gas-generating chemical compound or element. Preferred gas-generating compounds include citric acid and soda, Magnesium hydride (MgH₂), Calcium hydride (CaH₂), Magnesium dioxide (MgO₂), Magnesium powder, Aluminum powder, Lithium powder, and Calcium powder. Volatile fluid may be distributed or mixed into the chemical or compound substrate to make a slurry, paste, or other solidified form of volatile fluid.

An injection system, generally 164, is coupled to the bulk container 102 to deliver a chemical for combination with the solidized volatile fluid to cause a gas-generating chemical reaction. One workable injection system includes a reservoir 166 to hold water, and some sort of structure to introduce operable amounts of the water for combination with a gas-generating compound in the bulk container 102. An injector 164 may be manual or automatic, and can be mechanical (e.g. syringe, pump, solenoid), include various controls and/or electronics 110, and may include a device 168 structured to cause controlled release of a gas or fluid. Exemplary devices to cause controlled release of a fluid or gas are disclosed in U.S. Pat. Nos. 9,840,361 and 9,533,066, the entire contents of which are incorporated in this disclosure by reference.

One inherently spill-resistant form of volatile fluid may be characterized as a “solidified” volatile fluid article. In that case, a volatile fluid is dispersed in some way into a carrier material or substrate. An operable carrier material may be selected from the group including styrene-based polymers, styrene-based rubbers, EPDM, butadiene-based polymers, gum rubber, cellulosic rubber, thermoplastic polyether- or polyester-based polymers and rubbers (TPE), thermoplastic urethane (TPU), and combinations thereof, or an adsorbent material having high-surface area greater than about 10 m²/g, or an absorbent material including cellulose or polymer sponge, and gas-generating chemical compounds, (and a mixture of two or more of the foregoing elements). Generally, a scented or other volatile fluid is dispersed into the carrier material to a weight percent of between about 3% and about 400%, where weight percent is calculated as A/B*100, and A is weight of volatile fluid and B is weight of material. Desirably, the dispersion process is performed at ambient temperature conditions. Volatile fluids may include various scent-emitting oils, fragrances, mosquito repellant, and the like.

Besides the above-mentioned styrene- and butadiene-based polymers and such, a workable adsorbent material includes a high surface area (HSA) material selected from the group consisting of (high surface area ceramic, activated Alumina, γ-form Alumina, Silica, activated carbon, carbon black, molecular sieves, and zeolite). An adsorbent HSA material may be arranged in the form of a plurality of beads, powder, meal, or in any other desired shape. Desirably, certain HSA adsorbent materials have surface areas greater than about 10 m²/g.

A workable absorbent material includes cellulose or polymer (particularly sponge made from cellulose or polymer materials), fibrous or pulp organic material (e.g. wood, cotton), and the like. One currently preferred arrangement includes a meal manufactured from commercially available sponges by wetting a sponge, freezing the sponge, then shredding the sponge in a blender to form particles on the order of, or sized generally smaller than, a grain of rice, and drying the shredded material to form a meal-like power.

A carrier material may include an adsorbent constituent material in combination with an absorbent constituent material. A carrier material can sometimes be in the form of dough. For example, adsorbent material and absorbent material may initially be in powder and meal form, respectively, and can be mixed together with a volatile fluid to form a dough-like mass. One workable dough includes an adsorbent material component in powder or meal form, with the adsorbent constituent material having a surface area of greater than about 200 m2/g. Sometimes, a carrier material further includes hydrophobic material arranged to reduce a rate of discharge of volatile vapor from the carrier material to a local environment. Hydrophobic material may be provided as an exterior coating, or may be adsorbed into the carrier material.

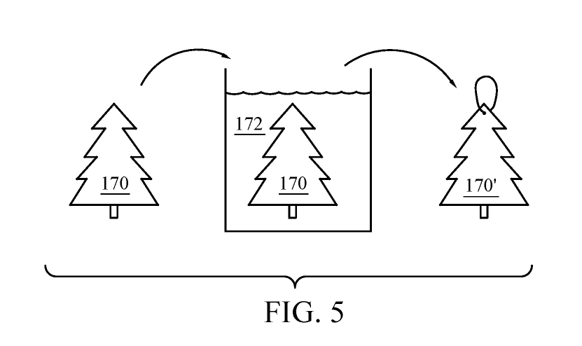

With reference to FIG. 5, a method of manufacturing a “solidified” volatile fluid article at ambient (or lower) temperature includes the steps of: forming a substrate 170; placing the formed substrate into contact with a volatile fluid 172 for a period of time to impregnate the substrate with a desired quantity of volatile fluid; and using the impregnated substrate 170′ as a spill-resistant emanator of volatile fluid. An emanator may include a volatile fluid that is dispersed into a substrate carrier material to a weight percent of between about 3%, 10%, 20%, 30%, etc., and up to about 150%, 200%, 300%, 400%, or more. Solidified volatile fluid articles may operate as emanators of volatile fluid at a desirable and humanly appreciable rate over an extended period of time.

To form a currently preferred emanator, a net-shape object may first be made from a carrier material substrate, such as a styrene-based or butadiene-based material, or TPE/TPU, or HSA material. The object can be any sort of 3-dimensional shape, such as a net-shape injection-molded ornamental figurine. The object is then soaked in, or otherwise wetted by, fragrant fluid at an ambient temperature, typically for a period of time greater than about 2 hours and generally less than about 4 days to form a volatile fluid-loaded carrier material, or “solidified” volatile fluid article. For purpose of this disclosure, “ambient temperature” may be about 20° C. (plus or minus perhaps about 2, 5, 7, 10, 15, or 20° C.), or even generally somewhere between about 0° C. and about 50° C.

In such an ambient temperature process, scented or other volatile fluid is typically dispersed into the material to a weight percent of between about 5% and about 100% to perhaps 200%, where weight percent is calculated as A/B*100, and A is weight of volatile fluid and B is weight of carrier material. In one embodiment, the weight gain of an exemplary styrene-based polymer or rubber-like material is about 35%. In preferred embodiments, the net-shape object may be characterized as non-porous, and the volatile fluid is adsorbed into the carrier material. However, certain carrier materials (or sometimes portions of a carrier material substrate assembly) may absorb the volatile fluid.

### EXAMPLES

A piece of styrene-butadiene rubber (SBR) weighing 6.198 grams was dipped into a sufficient quantity of citrus fragrance oil as to be fully submerged. After 12 hours at about 30° C., the SBR piece was removed, dried by paper towels, and weighed. The resulting weight was 14.688 grams. Therefore, the total weight gain was 8.49 grams. That constitutes a weight gain of over 100% at about 30° C. Then, the piece of SBR was placed into a bathroom having an approximately 120 ft2 floor, and the citrus smell filled the room and was initially strong. The citrus smell persisted for more than 30 days.

Various different types of SBR have been tested to evaluate the absorption capacity of the rubber. (It should be noted that SBS is also workable). The samples tested had different sources and different thicknesses. Pierced and unpierced samples were also tested. Seven different fragrances were used for absorption tests. The soak times varied from 3 to 6 days for various tests. However, it was established that 3 days was the adequate time period to achieve close to maximum absorption. Multiple tests were conducted at room temperature (72° F.) and weights were measured before and after the soak. The SBR used was 1/16″ thick, red in color and 70-75 A durometer. The results showed that different fragrances differed in their absorption limits. Test results have shown that that SBR has the capability to absorb 5 to 60% of its weight of fragrance.

It has been observed that the styrene portion of styrene-based materials can absorb fragrant oil and form a glue-like substance when exposed to liquid scented oil at a temperature between about 25° C. and about 50° C. A trigger event that appears to cause the phase transition between a solid polymer and a glue-like material is addition to the polymer of about 50% (by weight) of fragrance. Preferably, about 75% to 150% of the weight of the styrene-based material will be absorbed during the process to transform a solid polymer into a glue-like fragrant material.

One or more piece of styrene-based carrier or substrate material may be placed into a container, and a sufficient quantity of fragrant oil placed into contact with the substrate (e.g. to wet the carrier, and measured to provide essentially the desired amount of weight uptake). After a period of time, and in an ambient fluid temperature between about 25° C. and about 50° C. for a period of time greater than about four hours, the material can absorb a sufficient amount of oil as to change viscosity from a solid to a thick and viscous material. The resulting material may be characterized as a scent-emitting glue-like substance, and is very sticky. The glue-like material may then conveniently be applied as a coating to a substrate to form an air fresher or other emanator. Viscosity of the glue-like material is a function of the amount of fragrance absorbed by the base rubber, or rubber-like, material.

In one experiment, 1 g of polystyrene foam obtained from a crushed-up foam coffee cup was placed in a polypropylene cup. 1 g of fragrance was added to the polypropylene cup to bathe the crushed-up foam polystyrene. The fragrance was totally absorbed for a 100% weight gain. Although stirring was not part of the procedure, a viscosity change was detected at an estimated 75% weight gain. After about 4 hours, a fragrant glue was formed from the combination. The fragrant glue was very sticky, and would stick to any surface, especially porous surfaces like paper, cloth, etc. Furthermore, the fragrant glue appears to emit fragrance at a controlled rate. The fragrant glue-like substance was viscous, and would slowly extend in a drip-like extension from a stirring stick used to pick up the mixture. The thusly-formed fragrant glue was placed in a central container of an air freshener device; the air freshener device was placed in the sink of a bathroom; and fragrance level in the bathroom was monitored. The fragrance level was humanly appreciable and relatively constant for a period of time in excess of 18 days.

It has been observed that after losing 20-30% of the fragrance, the “stickiness” decreased. The resulting material then possessed a tacky property similar to a “post-it” note, or glue used to affix a removable object to a substrate. The object can then be removed without retaining residual adhesive, or the adhesive may be easily removed

It has also been observed that EPDM and Natural Gum rubbers may also absorb more than about 50% of their weight in fragrant oil, simply by submersion in fragrant oil at substantially ambient temperature for a sufficient length of time. Furthermore, cellulosic rubbers and TPU and TPE have been observed to operate in a similar manner.

Another embodiment according to certain principles of the instant invention includes high-surface area adsorbent materials that are loaded with a volatile fluid. For purpose of this disclosure, a high-surface area material is a material that exhibits greater than about 10 m2/g of surface area to weight or mass on the surface of the Earth. The fluid-loaded adsorbent materials may be employed directly as a stand-alone emanator, or may be disposed inside a container of some sort. Exemplary adsorbent materials include molecular sieves, zoolites, silica, silica gells, activated carbon, high surface area metal powders such as Iron, Magnesium, and activated Alumina, and the like.

A workable container may operate, for example, to maintain a collection of adsorbent material (e.g., in bead, pellet, chunk, or granular form, or in the form of a quantity of powder), in an organized state and in a desirably small volume. Certain containers simply look nice to a consumer. A currently preferred container is manufactured from a membrane, especially including a membrane formed from heat-shrinkable polymer material or nano-porous polymer material, wet cell battery separator, and the like. When present, the membrane desirably operates as an emanator. An emanator releases volatile fluid in vapor form into the local environment.

An exemplary embodiment includes a plurality of activated Alumina pellets disposed inside a container structured as a pouch formed by a vapor-porous, fluid-holding membrane, e.g., polymer material. The pellets may be pre-loaded with volatile fluid, or may be bathed in an excess quantity of volatile fluid inside the pouch. Preferred embodiments include heat-sealed ends. An advantage provided by certain pre-loaded pellets is that, in the event of rupture of the pouch, the volatile fluid is confined to the pellets, and will not cause a fluid spill and attendant stain of a surface on which the pellets fall.

An exemplary embodiment was formed from high-surface activated alumina pellets (beads) obtained from the Alfa-Aesar company. Alph Aesar Company has a web site located at world wide web alfa.com. The pellets were placed in a 300 degrees C. environment for a period of 24 hours to drive off any adsorbed moisture, then cooled to room temperature. 15 grams of fragrant volatile fluid was added to 20 grams of Alumina pellets at room temperature. A vacuum was then applied to the mixture to remove any adsorbed air from the pellets. It was observed that all of the volatile fluid was adsorbed by the pellets. The loaded pellets were then encased in polyolefin heat-shrinkable tubing exhibiting a 2:1 shrink ratio. Ends of the tube were heat-sealed.

Another embodiment was made by extracting high-surface area γ Alumina powder straight from a bottle, e.g., without additional processing to remove moisture. The particle size of the powder was between about 150 to about 200 microns. A total of five g of γ Alumina powder was added to 4 cc of fragrance (e.g., a volatile fluid). The Alumina soaked up all of the fragrance. The loaded powder was then disposed inside a polyolefin heat-shrinkable tubing exhibiting a 2:1 shrink ratio. The tube ends were sealed, and the sealed tubing formed an emanator to emit fragrance into the local environment.

Sometimes, a volatile fluid-loaded material may be, or include, one or more primarily absorbent material (absorbency typically being measured with respect to the volatile fluid). Absorption can be characterized as a bulk phenomenon. An absorbent material will take in or soak up (energy, or a liquid or other substance) by chemical or physical action, typically gradually. An absorbent material typically releases volatile fluid more rapidly to the environment than an adsorbent material, so for non-limiting example, can provide a strong initial burst of air freshening. In contrast, an adsorbent material holds molecules (e.g., of a gas, liquid, or solute) as a thin film on the outside surface or on internal surfaces within the material. Compared to an absorbent material, an adsorbent material tends to exhibit slower release, and at a more sustained rate, of volatile fluid over a longer period of time.

A workable absorbent material includes cellular foam, such as would be employed in manufacture of a cellulose or polymer sponge of the type that may be used to wash dishes, counters and kitchen surfaces, and the like. Other workable absorbent materials include paper, such as cellulose paper or porous polymer paper, other porous polymer materials, foams made from polymers including polystyrene and polybutadiene, polystyrene-based and polybutadiene-based rubber, plastic of various compositions and configurations, and other absorbent materials, matted or arranged fibrous material, cotton, and the like. Desirably, the absorbent material is inert to the volatile fluid, or at least exhibits a benign reaction when the two are in contact.

A volatile fluid-loaded material may include a mix of any suitable materials mentioned or suggested in this disclosure. Further, one or more such material may be carried in a matrix of other material, such as blended into a stream of plastic for injection molding. One or more additional beneficial agent may also be included, such as a selected commercially available enzymatic formulation for drain cleaning, or a gas-generating compound to promote transfer of volatile fluid vapor to the local environment. Several microbe-based enzymatic formulations are commercially available that are biodegradable. An exemplary gas-generating compound includes a metal carbonate with a solid acid, such as Calcium carbonate with citric acid. Moisture present in the local environment may be sufficient to generate gas from the gas-generating compound to facilitate, or enhance, delivery of volatile fluid vapor to that environment.

It has been found that powder made from a cellulose sponge can make a very desirable emanating structure. A commercially available cellulose sponge was soaked in water, frozen, and then crushed into a powder form in a blender. Remnant moisture in the thus-obtained and thawed foam powder may be removed prior to loading with a volatile fluid. For example, shredded or powdered foam may be dried by heating under vacuum for a period of time (e.g., 24 hours) at 60 to 80° C. The resulting absorbent foam powder, or meal, can be mixed with one or more adsorbent powder and a volatile fluid, like a fragrance, to make an emanating substance that resembles bread or cookie dough.

An exemplary emanating dough can be made according to the formula: 4 to 10 g cellulose foam powder; plus 10 to 30 g high surface area (>200 m2/g) γ Alumina powder; plus 25 to 70 ml of fragrant oil. Sometimes a solvent, such as acetone, may be included in the mix, as well as one or more other material(s) to accomplish a particular objective. For example, a hydrophobic material may sometimes be included to reduce rate of volatile fluid depletion from the carrier substrate.

A fragrance loading test was performed on a commercially available urinal screen manufactured from thermoplastic urethane (TPU). Embodiments of the TPU screen were soaked in a room temperature bath of fragrant oil (Marine Fresh) for different amounts of time. Typical processing temperature is ambient, or between about 0° C. to 50° C. The urinal screen included a screen element and a cup that fit into a hole, or receptacle, formed into the center of the screen. Test results are presented in Table 1 below. PCD is pitch circle diameter of the receptacle. Fitment is between the cup and the PCD. Initial fitment (OK) means the cup is secure in the screen. The TPU material swells as the TPU absorbs fragrance, causing a dimensional increase in the diameter of the cup receptacle. Eventually, the cup would not stay attached to the screen.

SBR, BBR, TPU, TPE, HSA and the like have been shown experimentally to be “thirsty” for fragrant oils, and can be used as efficient spill-resistant carriers and long-term emitters of volatile fluids. Volatile fluid carrying devices may be manufactured simply by impregnating net-shape pre-formed substrates using a low temperature adsorption and/or absorption process, and thereby avoid wasting large portions of volatile fluids during the manufacturing process. The “solidified” volatile fluid carried in such a substrate is generally spill-resistant. For example, a volatile fluid-loaded ceramic bead carrying 1 g of volatile fluid may be placed onto a sheet of paper, and will not leave a wet mark. In contrast, application of the same 1 g quantity of volatile fluid directly to the paper (comparable to a spill) will make a large visible wet mark on the paper.

TABLE 1

| | | | Initial | Final | |
|---|---|---|---|---|---|
| Screen | Soak Time | % increase | PCD | PCD | |
| No. | (hours) | in Wt | (mm) | (mm) | Fitment |
| 1 | 1 | 4.43 | 73.754 | 74.465 | OK |
| 2 | 2 | 5.57 | 73.754 | 75.114 | OK |
| 3 | 3 | 6.82 | 73.754 | 75.327 | Slightly |
| | | | | | loose |
| 4 | 4 | 8.17 | 73.754 | 75.636 | Slightly |
| | | | | | loose |
| 5 | 14 | 12.74 | 73.754 | 76.510 | Loose, |
| | | | | | could |
| | | | | | separate |
| 6 | 96 | 29.91 | 73.754 | 79.486 | Cannot |
| | | | | | hold cup |

While aspects of the invention have been described in particular with reference to certain illustrated embodiments, such is not intended to limit the scope of the invention. The present invention may be embodied in other specific forms without departing from its spirit or essential characteristics. For one example, one or more element may be extracted from one described or illustrated embodiment and used separately or in combination with one or more element extracted from one or more other described or illustrated embodiment(s), or in combination with other known structure. The described embodiments are to be considered as illustrative and not restrictive. Obvious changes within the capability of one of ordinary skill are encompassed within the present invention.

The scope of the invention for which a monopoly position is currently desired is indicated by the appended claims rather than by the foregoing description. All changes which come within the meaning and range of equivalency of the claims are to be embraced within their scope.

What is claimed is:

1. An apparatus, comprising:
a carrier material selected from the group consisting of styrene-based polymers, styrene-based rubbers, EPDM, butadiene-based polymers, gum rubber, cellulosic rubber, thermoplastic polyether- or polyester-based polymers and rubbers (TPE), thermoplastic urethane (TPU), high surface area ceramic, activated Alumina, activated carbon, zeolites, molecular sieves, or an adsorbent material having high-surface area greater than about 10 m²/g, an absorbent cellulose or polymer sponge, a gas-generating chemical compound, and a mixture of two or more of the foregoing elements; and
a volatile fluid dispersed inside the carrier material to a weight percent of between about 3% and about 400%, where weight percent is calculated as A/B*100, and A is weight of volatile fluid and B is weight of carrier material, to result in a spill-resistant solidified form of volatile fluid and to permit sustained release of volatile fluid vapor, wherein the volatile fluid is loaded into the carrier material at ambient temperature condition.

2. The apparatus according to claim 1, further comprising:
a volatile fluid chamber;
a quantity of volatile fluid-loaded carrier material disposed inside the volatile fluid chamber to permit sustained release of volatile fluid vapor to mix the vapor with a gas in the volatile fluid chamber;
a gas discharge port to communicate a mix of volatile fluid vapor and gas from the volatile fluid chamber to permit discharge of volatile fluid in vapor form into the local atmosphere; and
a gas-moving system to urge motion of gas from the volatile fluid chamber through the gas discharge port, wherein:
the gas-moving system comprises a compressor disposed upstream of the volatile fluid chamber.

3. The apparatus according to claim 1, further comprising:
a volatile fluid chamber;
a quantity of volatile fluid-loaded carrier material disposed inside the volatile fluid chamber to permit sustained release of volatile fluid vapor to mix the vapor with a gas in the volatile fluid chamber;
a gas discharge port to communicate a mix of volatile fluid vapor and gas from the volatile fluid chamber to permit discharge of volatile fluid in vapor form into the local atmosphere; and
a gas-moving system to urge motion of gas from the volatile fluid chamber through the gas discharge port, wherein:
the gas-moving system comprises a vacuum pump disposed downstream of the volatile fluid chamber.

4. The apparatus according to claim 1, further comprising:
a volatile fluid chamber;
a quantity of volatile fluid-loaded carrier material disposed inside the volatile fluid chamber to permit sustained release of volatile fluid vapor to mix the vapor with a gas in the volatile fluid chamber;
a gas discharge port to communicate a mix of volatile fluid vapor and gas from the volatile fluid chamber to permit discharge of volatile fluid in vapor form into the local atmosphere; and
a gas-moving system to urge motion of gas from the volatile fluid chamber through the gas discharge port, wherein:
the gas-moving system comprises a gas-generating material to increase a pressure inside the volatile fluid chamber as a result of a chemical reaction.

5. The apparatus according to claim 1, further comprising:
a volatile fluid chamber;
a quantity of volatile fluid-loaded carrier material disposed inside the volatile fluid chamber to permit sustained release of volatile fluid vapor to mix the vapor with a gas in the volatile fluid chamber;
a gas discharge port to communicate a mix of volatile fluid vapor and gas from the volatile fluid chamber to permit discharge of volatile fluid in vapor form into the local atmosphere; and
a gas-moving system to urge motion of gas from the volatile fluid chamber through the gas discharge port, wherein:
the gas-moving system comprises a one-way valve disposed in a path of gas to resist flow of gas in a direction away from the discharge port.

6. The apparatus according to claim 1, further comprising:
a volatile fluid chamber;
a quantity of volatile fluid-loaded carrier material disposed inside the volatile fluid chamber to permit sustained release of volatile fluid vapor to mix the vapor with a gas in the volatile fluid chamber;
a gas discharge port to communicate a mix of volatile fluid vapor and gas from the volatile fluid chamber to permit discharge of volatile fluid in vapor form into the local atmosphere; and
a gas-moving system to urge motion of gas from the volatile fluid chamber through the gas discharge port, wherein:
the gas-moving system comprises a transducer configured to operate on the solidified form to urge release of volatile vapor.

7. The apparatus according to claim 1, in combination with a gas-moving system constructed to produce periodic puffs of released gas to a local environment.

8. The apparatus according to claim 7, wherein:
the gas-moving system comprises a pressure relief valve.

9. The apparatus according to claim 4, wherein:
the gas-generating material comprises a substrate selected from the group consisting of citric acid and soda, Magnesium hydride, Calcium hydride, Magnesium dioxide, Magnesium powder, Aluminum powder, Lithium powder, and Calcium powder; and
the volatile fluid is dispersed into the substrate.

10. The apparatus according to claim 9, wherein:
the gas-generating system further comprises a water injection system configured and arranged to deliver water onto the substrate.

11. The apparatus according to claim 10, wherein:
the water injection system comprises a gas-generating cell arranged to increase pressure in a vessel of water for controlled delivery of the water to the substrate.

12. The apparatus according to claim 10, wherein:
the water injection system comprises a mechanical water injector.

13. The apparatus according to claim 6, wherein:
the transducer is selected from the group consisting of a heater, a nebulizer, and a microwave generator.

14. The apparatus according to claim 1, further comprising:
a volatile fluid chamber;
a quantity of volatile fluid-loaded carrier material disposed inside the volatile fluid chamber to permit sustained release of volatile fluid vapor to mix the vapor with a gas in the volatile fluid chamber;
a gas discharge port to communicate a mix of volatile fluid vapor and gas from the volatile fluid chamber to permit discharge of volatile fluid in vapor form into the local atmosphere; and
a gas-moving system to urge motion of gas from the volatile fluid chamber through the gas discharge port, wherein:
the volatile fluid chamber is structured as a replaceable cartridge to couple with structure associated with the gas-moving system.

15. The apparatus according to claim 1, further comprising:
a volatile fluid chamber;
a quantity of volatile fluid-loaded carrier material disposed inside the volatile fluid chamber to permit sustained release of volatile fluid vapor to mix the vapor with a gas in the volatile fluid chamber;
a gas discharge port to communicate a mix of volatile fluid vapor and gas from the volatile fluid chamber to permit discharge of volatile fluid in vapor form into the local atmosphere; and
a gas-moving system to urge motion of gas from the volatile fluid chamber through the gas discharge port, wherein:
the volatile fluid chamber is sized to hold volatile fluid in an amount between about 10 ml and about 1.5 liters.

16. A method, comprising:
disposing a quantity of volatile fluid in a substrate to form solidified volatile fluid; and
coupling the solidified volatile fluid to a gas-moving system for discharge of volatile vapor through a discharge orifice to a local environment.

17. The method according to claim 16, wherein:
the step of disposing volatile fluid in a substrate is performed at an ambient temperature.

18. The method according to claim 16, wherein:
the gas-moving system is operated at intervals to discharge discrete puffs of volatile fluid vapor to the local environment.

19. A method, comprising:
providing a volatile fluid distribution system comprising:
a volatile fluid chamber;
a volatile fluid disposed inside the volatile fluid chamber, the volatile fluid being configured and arranged in a solidified form to permit sustained release of volatile fluid vapor to mix the vapor with a gas in the volatile fluid chamber;
a gas discharge port to communicate a mix of volatile fluid vapor and gas from the volatile fluid chamber to permit discharge of volatile fluid in vapor form into the local atmosphere; and
a gas-moving system to urge motion of gas from the volatile fluid chamber through the gas discharge port; and
operating the volatile fluid distribution system to discharge volatile fluid vapor through the discharge port and into the local atmosphere.

20. The method according to claim 19, wherein:
the volatile fluid is dispersed into a substrate at ambient temperature conditions to form the solidified form of volatile fluid.
